(19) United States
(12) Patent Application Publication
(10) Pub. No.: US 2002/0177157 A1
(43) Pub. Date: Nov. 28, 2002

(54) Pairs of nucleic acid probes with interactive signaling moieties and nucleic acid probes with enhanced hybridization efficiency and specificity

(76) Inventors: Yuling Luo, Castro Valley, CA (US); Anthony C. Chen, Fremont, CA (US); Kaijun Li, Sunnyvale, CA (US)

Correspondence Address: Jill A. Jacobson, Morrison & Foerster LLP, 755 Page Mill Road, Palo Alto, CA, 94304-1018, US

(21) Appl. No.: 10/155,946

(22) Filed: May 24, 2002

Related U.S. Application Data
(60) Provisional application No. 60/293,666, filed on May 24, 2001.
(60) Provisional application No. 60/293,675, filed on May 24, 2001.

(51) Int. Cl. C12Q 1/68; C07H 21/04

(52) U.S. Cl. 435/6; 536/24.3

(57) ABSTRACT

The invention provides methods and kits for detecting and/or quantifying nucleic acid sequences of interest by using pairs of probes containing donor-acceptor moieties that when hybridized on a target polynucleotide, with one of the probes being hybridized to a sequence of interest in the target polynucleotide, places the donor-acceptor moieties in sufficiently close proximity such that a detectable signal is generated. Methods of the invention are particularly useful for genotyping analysis and gene expression profiling. Methods of the invention are easily adaptable to arrays and automation. The invention also provides probes with enhanced hybridization efficiency and/or specificity comprising a probe portion having a spacer element and/or a minor groove binder molecule, and methods and kits for using these probes.

This application claims the benefit of U.S. Provisional application Nos. 60/293,666, filed May 24, 2001, and 60/293,675, filed May 24, 2001, which are hereby incorporated by reference in their entirety for all purposes.

### TECHNICAL FIELD

The invention relates to methods for detecting and/or quantifying nucleic acid sequences and to methods for genotyping and expression profiling. The invention also relates to nucleic acid probes with enhanced hybridization efficiency and specificity and uses thereof.

### BACKGROUND OF INVENTION

The Human Genome Project has yielded millions of single nucleotide polymorphisms (SNPs), of which thousands can be used to identify genes involved in complex disease processes and to design individualized diagnostic and therapeutic strategies. Fulfilling this promise will require the genotyping of thousands of SNPs in thousands of samples efficiently and inexpensively.

A wide variety of technologies have been used to genotype SNPS, including differential oligonucleotide hybridization, single-base extension, oligonucleotide ligation, 5′ exonuclease assays such as TaqMan, molecular beacons, and Invader assays using a special endonuclease. These methods have been successfully used to genotype small numbers of SNPs at a time, but they are difficult to adapt to studies involving thousands of SNPs. Current genotyping methods either entail considerable effort in probe optimization or involve costly enzymatic steps. It has been extremely difficult to design a large set of oligonucleotide probes that have both similar Tm and enough allele-specific discriminating hybridization to distinguish single-base mismatches. For successful genotyping, extensive effort generally is required to optimize length and GC content of oligonucleotide probes. Without extensive probe optimization, differential oligonucleotide hybridization suffers from high false positive and false negative rates of base calling. In addition, enzymatic-based genotyping assays are inherently expensive and often are difficult to scale up, requiring extensive effort in sample preparation. Furthermore, most of the existing assays are inhomogeneous in nature, requiring labor-intensive separation steps to remove all unincorporated labeled nucleotides prior to detection. Some genotyping methods use a single oligonucleotide probe for allele-specific hybridization. Such methods suffer from low specificity, cannot handle the complexity of genomic DNA, and require pre-PCR amplification of regions containing the polymorphisms, a difficult and costly step. Thus, there is a need for an improved method for detecting and/or quantifying nucleic acid sequences of interest.

Nucleic acid probes immobilized on solid supports have been widely used for the detection of specific target sequences in solution samples. Probes have traditionally been immobilized on porous membranes such as nylon or nitrocellulose filters, which are accessible to binding of labeled targets in solution.

Recently, impermeable, rigid materials, such as glass, have been used for support of DNA molecules for hybridization. These materials have allowed miniaturization of the detection platform for applications such as DNA microarrays. Compared with porous membranes, where target molecules need to diffuse into pores to bind to probes, flat impermeable surfaces provide more immediate probe access and therefore higher hybridization efficiency.

Probes that are immobilized on these solid supports can be amplified PCR products of cDNAs or oligonucleotides that represent segments of genes of interest. Oligonucleotide probes provide advantages of easy material access, better quality control, and higher specificity. Methods for fabricating oligonucleotide arrays include in situ synthesis of oligonucleotides on solid supports and spotting of pre-synthesized oligonucleotides onto chemically functionalized surfaces. For cDNAs or pre-synthesized oligonucleotides, methods for attachment to solid supports include binding of the nucleic acid backbone to a solid surface or covalent attachment to the surface at one end of the oligonucleotide.

One of the key problems with solid supports is the accessibility of nucleic acid probes to targets in solution. Short oligonucleotides closely associated with a solid surface do not hybridize efficiently with targets in solution. Thus, there is a need for nucleic acid probes that exhibit improved hybridization efficiency and specificity for polynucleotide targets when attached to a solid support.

### SUMMARY OF THE INVENTION

The invention provides methods and kits for detecting and/or quantifying nucleic acid sequences of interest by using pairs of probes containing donor-acceptor moieties that when hybridized on a target polynucleotide, with one of the probes hybridized to a sequence of interest in the target polynucleotide, the donor-acceptor moieties are placed in sufficiently close proximity that a detectable signal is generated. Methods of the invention are particularly useful for genotyping analysis and gene expression profiling.

Accordingly, one aspect of the invention provides methods of detecting a sequence of interest in a sample. In one embodiment, said method comprises contacting the sample with an acceptor probe and a donor probe wherein (i) acceptor and donor probes comprise polynucleotides that are hybridizable to non-overlapping portions of a target polynucleotide, wherein the acceptor probe comprises a sequence hybridizable to the sequence of interest, (ii) said acceptor probe comprises an acceptor moiety and said donor probe comprises a donor moiety, and wherein hybridization of both the donor and acceptor probes to a target polynucleotide comprising the sequence of interest places the donor moiety and the acceptor moiety in an interaction mode capable of generating a detectable signal of a first intensity, (iii) a mismatch between the sequence of interest and the acceptor probe substantially prevents a portion of the acceptor probe from hybridizing to the sequence of interest such that the detectable signal generated by interaction of the donor moiety and the acceptor moiety is of a second intensity that is different (e.g., greater or less) than the first intensity, and wherein said portion of the acceptor probe is the portion that spans the sequence of the target polynucleotide between the mismatch site and hybridization site of the opposing end of the donor probe. In an embodiment, the invention provides a method of detecting a sequence of interest in a sample, said method comprising contacting the sample with an acceptor probe and a donor probe, wherein the acceptor and donor probes comprise polynucleotides that are hybridizable to non-overlapping portions of a target polynucleotide, wherein the acceptor probe comprises a sequence hybridizable to the sequence of interest, wherein said acceptor probe comprises an acceptor moiety and said donor probe comprises a donor moiety, and wherein hybridization of both the donor and acceptor probes to a target polynucleotide comprising the sequence of interest allows the donor moiety and the acceptor moiety to interact, wherein the interaction is detectable, and wherein a mismatch between the sequence of interest and the acceptor probe substantially prevents a portion of the acceptor probe from hybridizing to the sequence of interest such that the interaction between the donor and acceptor probe is diminished, and wherein said portion of the acceptor probe is the portion that spans the sequence of the target polynucleotide between the mismatch site and hybridization site of the opposing end of the donor probe. In another embodiment, the invention provides a method of detecting a sequence of interest in a sample, said method comprising contacting the sample with an acceptor probe and a donor probe, wherein the acceptor and donor probes are comprised within a single polynucleotide that is hybridizable to non-overlapping portions of a target polynucleotide, wherein the acceptor probe comprises a sequence hybridizable to the sequence of interest, wherein said acceptor probe comprises an attached acceptor moiety and said donor probe comprises an attached donor moiety, and wherein hybridization of both the donor and acceptor probes to a target polynucleotide comprising the sequence of interest allows the donor moiety and the acceptor moiety to interact, wherein the interaction is detectable, and wherein a mismatch between the sequence of interest and the acceptor probe substantially prevents a portion of the acceptor probe from hybridizing to the sequence of interest such that the interaction between the donor and acceptor probe is diminished, and wherein said portion of the acceptor probe is the portion that spans the sequence of the target polynucleotide between the mismatch site and hybridization site at which the donor moiety is attached.

In another embodiment, the invention provides methods of detecting a sequence of interest in a sample, said method comprising (a) contacting the sample with an acceptor probe and a donor probe wherein (i) the acceptor and donor probe comprise polynucleotides that are hybridizable to non-overlapping portions of a target polynucleotide, wherein the donor probe comprises a sequence hybridizable to the sequence of interest, (ii) said acceptor probe comprises an acceptor moiety and said donor probe comprises a donor moiety, and wherein hybridization of both the donor and acceptor probes to a target polynucleotide comprising the sequence of interest places the donor moiety and the acceptor moiety in an interaction mode capable of generating a detectable signal of a first intensity, (iii) a mismatch between the sequence of interest and the donor probe substantially prevents a portion of the donor probe from hybridizing to the sequence of interest such that the detectable signal generated by interaction of the donor moiety and the acceptor moiety is of a second intensity that is different (e.g., greater or less) than the first intensity, and wherein said portion of the donor probe is the portion that spans the sequence of the target polynucleotide between the mismatch site and hybridization site of the opposing end of the acceptor probe. In an embodiment, the invention provides a method of detecting a sequence of interest in a sample, said method comprising contacting the sample with an acceptor probe and a donor probe, wherein the acceptor and donor probes comprise polynucleotides that are hybridizable to non-overlapping portions of a target polynucleotide, wherein the donor probe comprises a sequence hybridizable to the sequence of interest, wherein said acceptor probe comprises an acceptor moiety and said donor probe comprises a donor moiety, and wherein hybridization of both the donor and acceptor probes to a target polynucleotide comprising the sequence of interest allows the donor moiety and the acceptor moiety to interact, wherein the interaction is detectable, and wherein a mismatch between the sequence of interest and the donor probe substantially prevents a portion of the donor probe from hybridizing to the sequence of interest such that the interaction of the donor moiety and the acceptor moiety is diminished, and wherein said portion of the donor probe is the portion that spans the sequence of the target polynucleotide between the mismatch site and hybridization site of the opposing end of the acceptor probe. In another embodiment, the invention provides a method of detecting a sequence of interest in a sample, said method comprising contacting the sample with an acceptor probe and a donor probe, wherein the acceptor and donor probes are comprised within a single polynucleotide that is hybridizable to non-overlapping portions of a target polynucleotide, wherein the donor probe comprises a sequence hybridizable to the sequence of interest, wherein said acceptor probe comprises an attached acceptor moiety and said donor probe comprises an attached donor moiety, and wherein hybridization of both the donor and acceptor probes to a target polynucleotide comprising the sequence of interest allows the donor moiety and the acceptor moiety to interact, wherein the interaction is detectable, and wherein a mismatch between the sequence of interest and the donor probe substantially prevents a portion of the donor probe from hybridizing to the sequence of interest such that the interaction of the donor moiety and the acceptor moiety is diminished, and wherein said portion of the donor probe is the portion that spans the sequence of the target polynucleotide between the mismatch site and hybridization site at which the acceptor moiety is attached.

In some embodiments, the method further comprises treating the sample such that existence of said interaction mode results in generation of said detectable signal. In some embodiments, the length of said portion of the probe that is substantially prevented from hybridizing to the sequence of interest is from about 3 to about 12 nucleotides. In other embodiments, the length of said portion of the probe that is substantially prevented from hybridizing to the sequence of interest is at least about 3, 6, 9, 12, or 15 nucleotides. In other embodiments, the length of said portion of the probe that is substantially prevented from hybridizing to the sequence of interest is 1, 2, 3, 4, 5, 6, 7, 8, 9, 10, 11, 12, 13,14, or 15 nucleotides.

In some embodiments, the donor and acceptor probes are separate polynucleotides. In other embodiments, the donor and acceptor probes are part of a single polynucleotide, wherein the polynucleotide comprises sequences that are hybridizable to non-overlapping portions of a target polynucleotide, wherein a sequence comprising either a donor moiety or an acceptor moiety is hybridizable to a sequence of interest and a sequence comprising the counterpart moiety (i.e., donor is counterpart to acceptor, and acceptor is counterpart to donor) is hybridizable to a non-overlapping portion of the target such that when the sequences containing both the donor and acceptor moieties are hybridized to the target, the donor and acceptor moieties interact in a detectable manner, and wherein a mismatch between the sequence of interest and the donor probe substantially prevents a portion of the donor probe from hybridizing to the sequence of interest such that the interaction of the donor moiety and the acceptor moiety is diminished. In some embodiments in which the donor and acceptor probes are part of the same polynucleotide, the polynucleotide is at least about 25-200, 50-150, or 60-100 nucleotides in length, and in some embodiments, the polynucleotide is about 25, 50, 60, 80, 100, 120, 140 150, 160, 180 or 200 nucleotides in length.

In some embodiments of methods of the invention, the sequence of interest contains a mutation. In some embodiments wherein the sequence of interest contains a mutation, said mutation is selected from the group consisting of a point mutation, a deletion and an insertion. In some embodiments wherein the sequence of interest contains a mutation that is a deletion, said deletion is of fewer than about 8, 5 or 3 nucleotides. In some embodiments wherein the sequence of interest contains a mutation that is an insertion, said insertion is of fewer than about 8, 5 or 3 nucleotides. In some embodiments of the methods of the invention, the sequence of interest contains a single nucleotide polymorphism.

In some embodiments of methods of the invention, the acceptor and donor moieties are capable of fluorescence resonance energy transfer. For example, the moieties can be fluorescent molecules, or fluorescent molecules and quenchers. In other embodiments, the acceptor and donor moieties are different portions of an enzyme, such that when both moieties are in close enough proximity, the enzyme is capable of catalysis of a substrate.

In other embodiments of methods of the invention, the acceptor and donor moieties are located at opposing ends of the probes when hybridized to the target polynucleotide. In some of these embodiments, the ends are the 5′ end of one probe and the 3′ end of the other probe.

In other embodiments of the methods of the invention, the length of said portion of the probe that is substantially prevented from hybridizing to the sequence of interest is from about 3 to about 15 nucleotides.

In some embodiments of methods of the invention, probes are provided as an array. In some of these embodiments, the acceptor probe is attached to a solid support and the donor probe is provided in a solution phase. In some of these embodiments, the solid support is provided as a 3-dimensional internal probe carrier for binding a target molecule to a probe comprising a solid support having a first and a second surface, at least one discrete throughwell on the solid support, the well comprising an elongated bore structure traversing the solid support from the first to the second surface and defined by at least one inner side wall, wherein each well is individually identifiable by its position on the solid support, and at least one acceptor probe is attached to an inner side wall of the well. In other embodiments wherein probes are provided as an array, the donor probe is attached to a solid support and the acceptor probe is provided in a solution phase. In some of these embodiments, the solid support is provided as a 3-dimensional internal probe carrier for binding a target molecule to a probe comprising a solid support having a first and a second surface, at least one discrete throughwell on the solid support, the well comprising an elongated bore structure traversing the solid support from the first to the second surface and defined by at least one inner side wall, wherein each well is individually identifiable by its position on the solid support, and at least one donor probe is attached to an inner side wall of the well.

In yet another aspect of the invention, methods of quantifying a sequence of interest in a sample are provided, said methods comprising quantifying the detectable signal generated in the methods described in the preceding paragraphs.

In still another aspect, the invention provides methods of determining a gene expression profile in a sample, said methods comprising (a) detecting the presence of two or more sequences of interest in said sample using any of the methods of the invention described in the preceding paragraphs; and (b) quantifying the detectable signal that is generated for each of said sequences of interest to determine an amount of each sequence of interest present in said sample.

In yet another aspect, the invention provides kits for characterizing a sequence of interest, comprising (a) an acceptor probe and a donor probe wherein (i) the acceptor and donor probe comprise polynucleotides that are hybridizable to non-overlapping portions of a target polynucleotide, wherein the acceptor probe comprises a sequence hybridizable to the sequence of interest, (ii) said acceptor probe comprises an acceptor moiety and said donor probe comprises a donor moiety, and wherein hybridization of both the donor and acceptor probes to a target polynucleotide comprising the sequence of interest places the donor moiety and the acceptor moiety in an interaction mode capable of generating a detectable signal of a first intensity, (iii) a mismatch between the sequence of interest and the acceptor probe substantially prevents a portion of the acceptor probe from hybridizing to the sequence of interest such that the detectable signal generated by interaction of the donor moiety and the acceptor moiety is of a second intensity that is different (e.g., greater or less) than the first intensity, and wherein said portion of the acceptor probe is the portion that spans the sequence of the target polynucleotide between the mismatch site and hybridization site of the opposing end of the donor probe. In an embodiment, the invention provides a kit for characterizing a sequence of interest, comprising an acceptor probe and a donor probe, wherein the acceptor and donor probes comprise polynucleotides that are hybridizable to non-overlapping portions of a target polynucleotide, wherein the acceptor probe comprises a sequence hybridizable to the sequence of interest, wherein said acceptor probe comprises an acceptor moiety and said donor probe comprises a donor moiety, and wherein hybridization of both the donor and acceptor probes to a target polynucleotide comprising the sequence of interest allows the donor moiety and the acceptor moiety to interact, wherein the interaction is detectable, and wherein a mismatch between the sequence of interest and the acceptor probe substantially prevents a portion of the acceptor probe from hybridizing to the sequence of interest such that the interaction between the donor moiety and the acceptor moiety is diminished, and wherein said portion of the acceptor probe is the portion that spans the sequence of the target polynucleotide between the mismatch site and hybridization site of the opposing end of the donor probe.

In another aspect, the invention provides kits for characterizing a sequence of interest comprising (a) an acceptor probe and a donor probe, wherein (i) the acceptor and donor probe comprise polynucleotides that are hybridizable to non-overlapping overlapping portions of a target polynucleotide, wherein the donor probe comprises a sequence hybridizable to the sequence of interest, (ii) said acceptor probe comprises an acceptor moiety and said donor probe comprises a donor moiety, and wherein hybridization of both the donor and acceptor probes to a target polynucleotide comprising the sequence of interest places the donor moiety and the acceptor moiety in an interaction mode capable of generating a detectable signal of a first intensity, (iii) a mismatch between the sequence of interest and the donor probe substantially prevents a portion of the donor probe from hybridizing to the sequence of interest such that the detectable signal generated by interaction of the donor moiety and the acceptor moiety is of a second intensity that is different (e.g., greater or less) than the first intensity, and wherein said portion of the donor probe is the portion that spans the sequence of the target polynucleotide between the mismatch site and hybridization site of the opposing end of the acceptor probe. In an embodiment, the invention provides a kit for characterizing a sequence of interest comprising an acceptor probe and a donor probe, wherein the acceptor and donor probes comprise polynucleotides that are hybridizable to non-overlapping portions of a target polynucleotide, wherein the donor probe comprises a sequence hybridizable to the sequence of interest, wherein said acceptor probe comprises an acceptor moiety and said donor probe comprises a donor moiety, and wherein hybridization of both the donor and acceptor probes to a target polynucleotide comprising the sequence of interest allows the donor moiety and the acceptor moiety to interact, wherein the interaction is detectable, wherein a mismatch between the sequence of interest and the donor probe substantially prevents a portion of the donor probe from hybridizing to the sequence of interest such that the interaction between the donor moiety and the acceptor moiety is diminished, and wherein said portion of the donor probe is the portion that spans the sequence of the target polynucleotide between the mismatch site and hybridization site of the opposing end of the acceptor probe.

In kits of the invention, probes are provided in single or separate packaging, for example in a single reaction vessel or in separate reaction vessels. Kits of the invention can further comprise instructions and/or other components (such as appropriate buffers) for using methods of the invention to characterize nucleic acid sequences of interest.

In another aspect, the invention provides compositions comprising any of the components (such as donor-acceptor probes) and/or reaction mixtures used in carrying out methods of the invention, and/or any complex(es) formed in carrying out these methods.

The invention also provides probes with enhanced hybridization or association efficiency and specificity when attached to a solid support. These probes have a spacer component between a probe portion (e.g. a nucleic acid, protein, or cell) and a solid substrate and/or a minor groove binding molecule where the probe portion is a nucleic acid. Methods and kits for using these probes, for example to characterize nucleic acid sequences of interest, are also provided.

Accordingly, in one aspect, the invention provides a nucleic acid probe comprising a nucleic acid, a spacer and a minor groove binder (MGB), wherein one end of the spacer is linked to the nucleic acid, preferably at one end or near one end of the nucleic acid, and the other end of the spacer is linked to a solid substrate. In some embodiments, the MGB is linked to an end or near an end of the nucleic acid. In other embodiments, the MGB is linked to an internal nucleotide preferably at least about 3, more preferably at least about 5, even more preferably at least about 10, still more preferably at least about 20, and even more preferably at least about 30 nucleotides from an end of the nucleic acid.

In another aspect, the invention provides a nucleic acid probe comprising a nucleic acid, a spacer and a minor groove binder, wherein one end of the spacer is linked to the nucleic acid at one end or near one end of the nucleic acid and the other end of the spacer is linked to a solid substrate, and wherein the MGB is linked to the other end or near the other end of the nucleic acid. In one embodiment, a nucleic acid probe comprising a nucleic acid with first and second ends, a spacer with first and second ends, and a minor groove binder (MGB), wherein the first end of the spacer is linked to the nucleic acid at or near the first end of the nucleic acid, the second end of the spacer is linked to a solid substrate, and the MGB is linked to the nucleic acid at or near the second end of the nucleic acid.

In yet another aspect, the invention provides a nucleic acid probe comprising a nucleic acid, a spacer, and a minor groove binder, wherein one end of the spacer is linked to the 5′ end of the nucleic acid and the other end of the spacer is linked to a solid substrate, and wherein the MGB is linked to the 3′ end of the nucleic acid.

In still another aspect, the invention provides a nucleic acid probe comprising a nucleic acid, a spacer, and a minor groove binder, wherein one end of the spacer and the MGB are linked to the nucleic acid at the same end or near the same end of the nucleic acid, and the other end of the spacer is linked to a solid substrate.

In some embodiments, the invention provides a probe or an array of probes having a probe portion (such as a nucleic acid) and a spacer portion that can be attached to a substrate to form the array of probes. In one preferred embodiment, the spacer comprises ethylene glycol. In some embodiments, the spacer is ethylene glycol. In other embodiments, the spacer is a polymer containing ethylene glycol. In still other embodiments, the spacer is a polyethylene glycol polymer selected from the group consisting of polyethylene glycol molecules having at least about 3, 6, 9, 12, 18, or 25 ethylene glycol monomers. Preferably the probe portion has an MGB attached to the probe portion away from the point where the spacer portion is attached, and preferably the MGB is attached at or near a free end of the probe portion when the probe is attached to a substrate.

In embodiments as described above, the spacer is linked to the nucleic acid by a chemically active functional group. The chemically active functional group can be selected from the group consisting of acetyl, phosphate, carboxyl, amino or thiol. In some embodiments, a unit containing the spacer linked to the nucleic acid is pre-synthesized (prior to attaching to solid support) using methods such as phosphoamidite chemistry. In other embodiments, the spacer is generated as part of a solid support surface, wherein the spacer has a functional group at its extremity which allows attachment (such as covalent attachment) of nucleic acids to the spacer (the nucleic acid may or may not have an MGB attached).

In some embodiments as described above, the spacer of the probes of the invention comprises (deoxyribothymidine)ₙ, wherein n=1−50.

In embodiments as described above, the MGB in the probes of the invention is selected from the group consisting of an antibiotic and a synthetic molecule.

In embodiments as described above, the nucleic acid in the probes of the invention is selected from the group consisting of deoxyribonucleic acids (DNA), ribonucleic acids (RNA), DNA-RNA hybrid polynucleotides, synthetic polynucleotides, and oligonucleotides. The nucleic acid of the probes of the invention is preferably an oligonucleotide. In some embodiments, the nucleic acid of the probes of the invention comprises a label.

In embodiments as described above, the solid substrate on which the spacer of probes of the invention is immobilized is provided by an apparatus comprising a flexible elongated substrate having a first substrate surface, a length, and a width; and a plurality of non-identical probes immobilized on discrete areas of a probe-containing portion of the substrate surface, at least one of said discrete areas containing a probe of the invention.

In embodiments as described above, the solid substrate on which probes (i.e., the spacer of probes) of the invention is immobilized is provided by an apparatus comprising a flexible elongated substrate having a substrate surface, a length, and a width; a first layer on the surface of the substrate; and a plurality of non-identical probes immobilized on a probe-containing portion of the surface of said layer, said probe-containing portion having a length and a width such that the ratio of the length of the probe-containing portion to the width of the probe-containing portion exceeds 5:1.

In embodiments as described above, the solid substrate on which the spacer of probes of the invention is immobilized are provided by an apparatus comprising a flexible substrate having at least a first surface; and a plurality of probes immobilized on the first surface of the substrate and arranged in a single-file row at a linear density exceeding 50 probes/linear cm, wherein one of said plurality of probes is a probe of the invention.

In embodiments as described above, the solid substrate on which the spacer of probes of the invention is immobilized is provided by an apparatus comprising a probe-carrying tape apparatus that is configured to bind samples to form sample-probe complexes, said tape comprising (a) a flexible tape substrate having a thickness not exceeding about 500 micrometers, and having a surface; and (b) a plurality of non-identical probes immobilized on discrete areas of a probe-containing portion of the substrate surface, each of said discrete areas containing one probe, wherein at least one of said probes is a probe of the invention.

In another aspect, the invention provides methods for enhancing hybridization efficiency or specificity of a probe, said methods comprising (a) inserting a spacer between a nucleic acid and a solid substrate on which the spacer is immobilized; and (b) incorporating a minor groove binding molecule into the nucleic acid.

In another aspect, the invention provides methods of characterizing a sequence of interest in a sample, said methods comprising (a) contacting said sample with a nucleic acid probe of the present invention; and (b) detecting hybridization of said nucleic acid probe to said sequence of interest.

In yet another aspect, the invention provides methods of making a nucleic acid probe with enhanced hybridization efficiency and specificity, said method comprising (a) linking a spacer to a nucleic acid, wherein said spacer is used for linking said nucleic acid to a solid substrate, and (b) incorporating a minor groove binder molecule into the nucleic acid.

In still another aspect, the invention provides kits and compositions for characterizing a sequence of interest in a sample, said kits and compositions comprising one or more of the probes of the invention. The kits can further comprise instructions and/or other components (such as appropriate buffers) for using the probes in characterizing nucleic acid sequences of interest.

### BRIEF DESCRIPTION OF THE DRAWINGS

FIG. 1 shows some of the advantages of methods of the invention by comparison to single-probe hybridization methods.

FIG. 2 shows some of the advantages of methods of the invention in the context of gene expression profiling by comparison to single-probe hybridization methods.

FIG. 3 is a schematic depiction of one embodiment of methods of the invention, showing interaction of FRET moieties in the presence and absence of a mismatch between a probe and a sequence of interest.

FIG. 4 is a schematic depiction of one embodiment of methods of the invention performed in array format.

FIG. 5 illustrates genotyping results in array format.

FIG. 6 depicts probes used in experiments to assess the interaction between a FRET probe pair.

FIG. 7 depicts the results of interaction between the probes shown in FIG. 6 at different temperatures.

FIG. 8 is a schematic illustration of two probes linked to a solid support through a spacer. One probe is linked through a PEG linker as the spacer and the other probe is linked through a deoxyribothymidine linker as the spacer.

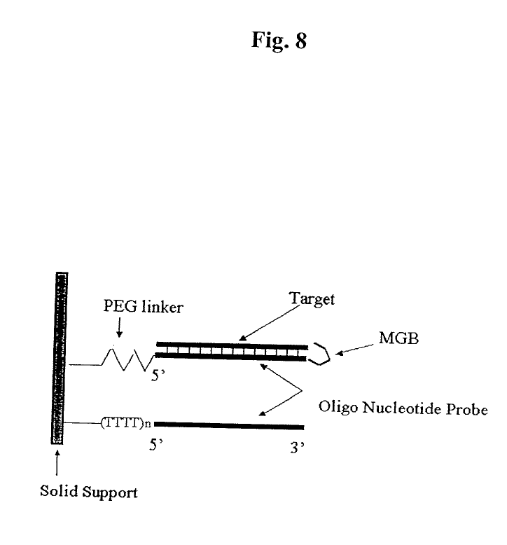

FIG. 9 shows the increase in hybridization intensity when PEG linkers or oligo (dT) linkers are used to increase the distance between a hybridization probe and the surface of a support. Values (Y axis, artificial units) are averaged from two genes (Dap and GAPDH) and five hybridization conditions (10-50% formamide). When each probe was observed separately, similar trends of signal intensity change were observed. 30-bare or 50-bare: probe directly linked to support without spacer; 30-T: 30-mer probes linked to a support with 20-residue dT spacer; 50-T: 50-mer probes linked to a support with 10-residue dT spacer; 30-IX and 50-1X: 30 or 50-mer probes linked to a support with one PEG oligomer composed of 18 residues; 30-2X and 50-2X: 30 or 50-mer probes linked to a support with two PEG oligomers each composed of 18 residues (a total of 36 residues); 30-3X and 50-3X: 30 or 50 mer probes linked to a support with three PEG oligomers each composed of 18 residues (a total of 54 residues).

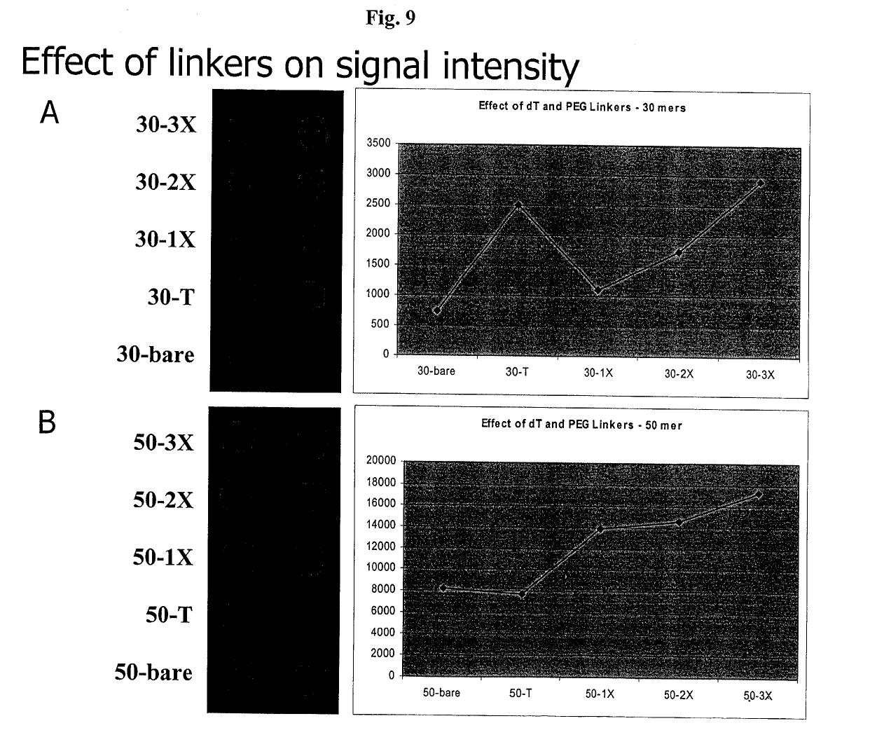

### MODES FOR CARRYING OUT THE INVENTION

We have discovered methods for detecting and/or quantifying nucleic acid sequences of interest by using pairs of probes with interactive signaling entities. Methods of the invention include pairs of probes containing a donor moiety on one member of the pair and an acceptor moiety on the other member of the pair, and include hybridizing a pair of probes to a target polynucleotide such that a detectable signal is generated when one of the probes hybridizes to a sequence of interest, placing donor and acceptor moieties in sufficiently close proximity to each other to generate the signal. Generation and intensity of the signal indicates hybridization of a probe to the sequence of interest, thus indicating (and enabling the quantification of) the presence of the sequence of interest. The methods of the invention enhance specificity of probe hybridization and thus accuracy of sequence detection and/or quantification by using probes designed such that, where there is a mismatch between a probe and a sequence of interest, the probe, or a portion of the probe, does not hybridize, wherein the lack of hybridization reduces the degree of or prevents an interaction mode between the donor-acceptor moieties of the probes.

These methods can be used in a variety of applications for nucleic acid analysis, including genotyping thousands of SNPs in parallel. These methods require minimal effort in probe optimization and do not involve costly enzymatic steps. In addition, the assays can be homogeneous in nature and adaptable to automation. Furthermore, the assays are highly specific and could potentially be conducted with general amplification of genomic DNA rather than requiring pre-analysis PCR amplification of specific nucleic acid sequences of interest.

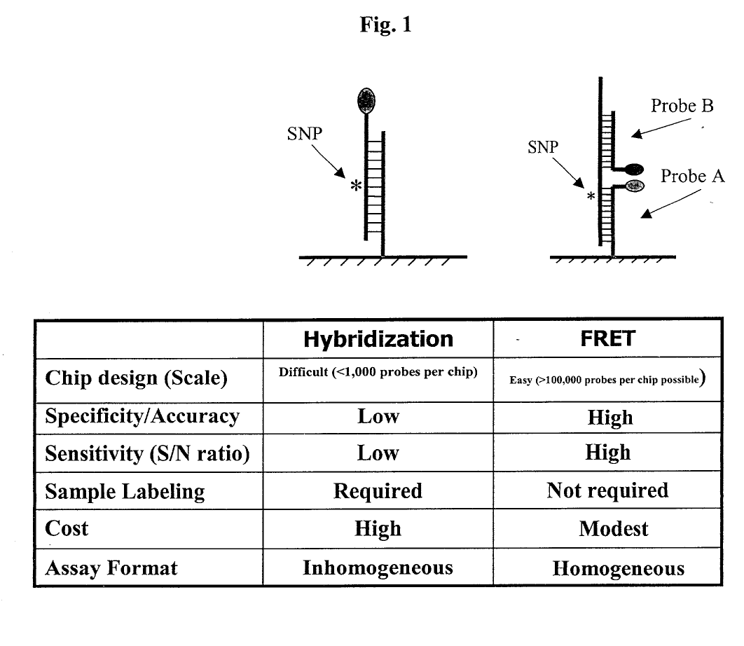

Methods of the invention are particularly useful for genotyping and gene expression profiling. These methods offer various advantages over other methods. Some of the advantages of genotyping methods of the invention, such as those based on FRET (Fluorescence Resonance Energy Transfer), are listed in FIG. 1. For example, in a hybridization-based method, the target needs to be labeled, most commonly with fluorescent dyes. Such labeling limits the complexity of the target pool, and only a small number of target SNPs can be analyzed simultaneously. In addition, the use of a single probe limits the specificity of the signal, particularly when highly complex targets are used, due to cross-hybridizations. On the other hand, a FRET-based assay does not require labeling of the target, thereby eliminating a complex and expensive step. Using some of the newly-developed target amplification methods, such as rolling cycle amplification (RCA), a large amount of target DNA can be obtained for such FRET assays without the use of expensive gene-specific PCR amplification methods. In addition, since two probes must both hybridize to the target molecule in order to generate a FRET signal, the specificity of the signal is greatly increased. Unlike hybridization-based assays, a FRET assay does not require washing of unbound probe before signal acquisition, since unhybridized acceptor probes will not generate signals when the exciting wavelength is limited to that of the donor probe. This makes it possible to monitor the hybridization process in real time, using either a scanner equipped with a temperature control module, or a system similar to real-time PCR.

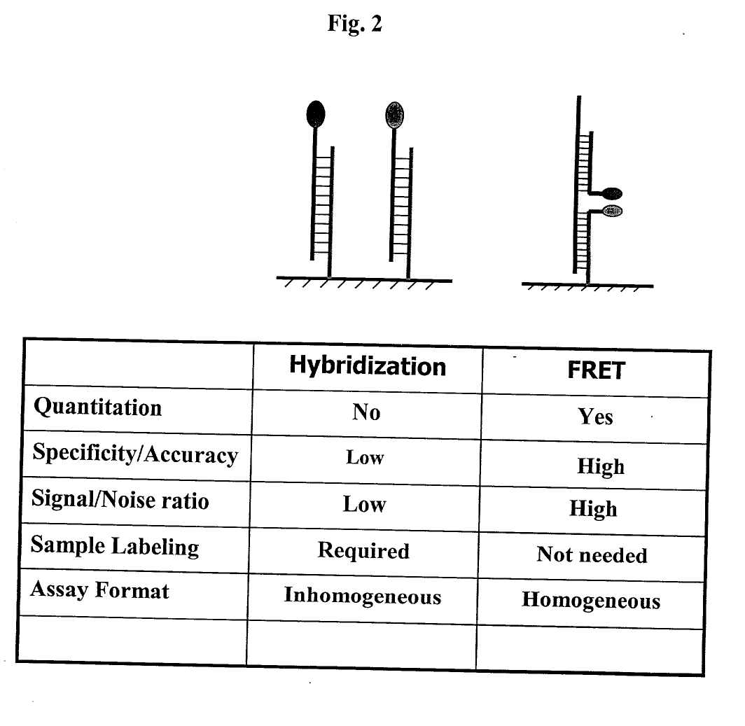

Some of the advantages of expression profiling methods of the invention are listed in FIG. 2, particularly with respect to an array format, where thousands of genes can be interrogated. Advantages listed for FRET-based genotyping, including high specificity, high signal to noise ratio, elimination of washing and target-labeling steps, are similar to advantages for FRET-based gene profiling. Specifically, the elimination of probe labeling dramatically reduces the workload of array users and removes a significant source of variation in microarrray hybridization. In addition, since the amount of capture probe on the array can be quantified using its fluorescent label, quantitation of the target is also possible when one compares the FRET signal with the capture probe signal.

General Techniques

The practice of the present invention may employ, unless otherwise indicated, conventional techniques of molecular biology (including recombinant techniques), microbiology, cell biology, biochemistry, and immunology, which are within the skill of the art. Such techniques are explained filly in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Current Protocols in Molecular Biology” (F. M. Ausubel et al., eds., 1987, and periodic updates); “PCR: The Polymerase Chain Reaction”, (Mullis et al., eds., 1994).

Primers, oligonucleotides and polynucleotides employed in the present invention can be generated using standard techniques known in the art.

Definitions

The term “probe,” as used herein, refers to a nucleic acid molecule or a set of copies of a nucleic acid molecule which is capable of specific binding to a target nucleic acid molecule(s) in a particular sample or portion of a sample. The set may contain any number of copies of the nucleic acid molecule. “Probes,” as used herein, refers to more than one such set of molecules. Probes may be immobilized on a substrate by either covalent or noncovalent attachment. “Nucleic acid,” or “polynucleotide,” as used interchangeably herein, refer to polymers of nucleotides of any length, and include DNA, RNA, PNA and hybrids thereof. The nucleotides can be deoxyribonucleotides, ribonucleotides, modified nucleotides or bases, and/or their analogs, or any substrate that can be incorporated into a polymer by DNA or RNA polymerase. A nucleic acid may comprise modified nucleotides, such as methylated nucleotides and their analogs. If present, modification to the nucleotide structure may be imparted before or after assembly of the polymer. The sequence of nucleotides may be interrupted by non-nucleotide components. A nucleic acid may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping groups moieties of from about 1 to about 20 carbon atoms. Other hydroxyls may also be derivatized to standard protecting groups. Polynucleotides can also contain analogous forms of ribose or deoxyribose sugars that are generally known in the art, including, for example, 2-O-methyl-, 2-O-allyl, 2-fluoro- or 2-azido-ribose, carbocyclic sugar analogs, α-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S(“thioate”), P(S)S (“dithioate”), “(O)NR₂(“amidate”), P(O)R, P(O)OR′, CO or CH₂ (“formacetal”), in which each R or R′ is independently H or substituted or unsubstituted alkyl (about 1- about 20 C) optionally containing an ether (—O—) linkage, aryl, alkenyl, cycloalkyl, cycloalkenyl or araldyl. Not all linkages in a nucleic acid need be identical. The preceding description applies to all nucleic acid molecules referred to herein, including RNA and DNA.

“Acceptor probe,” as used herein, is a probe that contains an acceptor moiety. An “acceptor moiety,” as used herein, refers to the moiety of a interactive pair of acceptor-donor moieties that in the presence of the donor moiety is rendered capable of being detected. A “donor probe,” as used herein, is a probe containing a donor moiety. As used herein, “donor moiety” refers to the moiety of an interactive pair of donor-acceptor moieties that when present in sufficient proximity to its counterpart acceptor moiety renders the acceptor moiety capable of being detected, through for example, fluorescence, or a colorimetric product of a reaction. A “detectable signal,” as used herein, refers to any signal than can be detected by methods known in the art, including by naked eye and with the aid of an instrument. There is a detectable signal when there is an identifiable characteristic that indicates the proximity of a donor moiety and an acceptor moiety to each other. An identifiable characteristic can be generation of a signal (such as color or light) or reduction (including quenching) of a signal (such as when proximity of a donor-acceptor moiety pair results in the quenching of signal normally generated by an acceptor moiety when it is not in sufficient proximity with the donor moiety). The moieties of a donor-acceptor pair are preferably capable of resonance energy transfer, such as fluorescence resonance energy transfer. Acceptable fluorophore pairs for use as fluorescent resonance energy transfer pairs are well known in the art and include, but are not limited to, fluorescein/rhodamine, phycoerythrin/Cy7, fluorescein/Cy5 or fluorescein/Cy5.5. Others are described in, for example, U.S. Pat. No. 4,996,143 and U.S. Pat. No. 5,688,648. The moieties of a donor-acceptor pair can also be an enzyme and its corresponding substrate or two different portions or subunits of an enzyme that catalyze a detectable reaction, such that when both moieties are in close enough proximity, the enzyme is capable of catalysis of a substrate and when the moieties are not in close enough proximity for catalysis, a diminished amount of detectable reaction product is produced.

A “probe that spans a sequence of interest,” or variations thereof, as used herein, refers to a probe that is capable of hybridizing to the sequence of interest if there is no mismatch between the probe and sequence of interest. A “co-hybridized probe,” as used herein, generally refers to a probe that is hybridizable to a site in the target polynucleotide near or adjacent to the target hybridization site of the other probe that spans a sequence of interest, such that the donor and acceptor moieties of the two hybridized probes are capable of interacting, wherein the co-hybridized probe contains the counterpart moiety (to the probe that spans a sequence of interest) in the donor-acceptor moiety pair.

A “sequence of interest,” as used herein, is a nucleic acid sequence the detection or quantification of which is desired. The identity of a sequence of interest is generally known. However, in some embodiments of the invention, the sequence of interest is unknown. As used herein, a sequence of interest can be a single nucleotide base or more than a single nucleotide base. A sequence of interest can be a known polymorphic sequence, including, for example, single nucleotide polymorphism.

A “target polynucleotide,” as used herein, is a polynucleotide known or suspected to comprise a sequence of interest.

“A donor moiety and an acceptor moiety are placed in an interaction mode capable of generating a detectable signal,” and variations thereof, as used herein, refer to placement of the moieties of a donor-acceptor pair in sufficient proximity that the moieties are capable of interacting to render the acceptor moiety capable of being detected. In some embodiments, an additional component may be added to complete detection. The interaction mode can result in an on/off phenomenon wherein the acceptor moiety is rendered capable of generating said signal when the donor-acceptor moieties are positioned less than a particular distance from each other, but not if positioned more than said distance from each other. The interaction mode can also result in a gradation of signal intensity that is generated as a proportion of distance of the moieties from each other, as discussed below.

The phrase “substantially prevents a portion of the probe from hybridizing,” refers to a substantial inhibition of a portion of a probe that has a mismatch with a sequence of interest in a target polynucleotide from hybridizing to said sequence, such that the donor and acceptor moieties are not in close enough proximity for the acceptor moiety to be detectable. There is substantial inhibition when there is a reduction of preferably at least about 50%, more preferably at least about 70%, even more preferably at least about 90%, still more preferably at least about 95%, and most preferably 100% (i.e. no hybridization) reduction in hybridization of the portion of the probe to its complementary sequence in the target polynucleotide.

“Hybridization,” as used herein, refers to association between two single-stranded polynucleotides to form a duplex via hydrogen bonding. As used herein, hybridization includes mismatches between two single-stranded polynucleotides that are associated through hydrogen bonding for greater than a transient period. Optimal hybridization conditions depend on a variety of factors, including the length and base compositions of the polynucleotides, the extent of base mismatching between the two polynucleotides, the presence of salt and organic solvents, polynucleotide concentration, and temperature. Generally, the higher the “stringency” of the hybridization conditions, the higher the sequence identity must be between two polynucleotides to allow them to hybridize. Appropriate hybridization conditions of varying stringency are widely known and published in the art (see, for example, Sambrook et al. (2001), “Molecular Cloning: A Laboratory Manual, third edition). Generally, high stringency hybridization conditions may be selected at about 5° C. lower than the thermal melting point (Tm) for a specific double-stranded sequence at a defined ionic strength and pH. The Tm is the temperature (under defined ionic strength and pH conditions) at which 50% of a polynucleotide sequence hybridizes to a perfectly matched (i.e., complementary) sequence. Typically, stringent conditions will be those in which the salt concentration is at least about 0.02 molar at pH 7 and the temperature is at least about 60° C. As other factors may significantly affect the stringency of hybridization, including, for example, nucleotide base composition and size of the complementary strands, the presence of organic solvents, salt, formamide, DMSO, or glycerol, and the extent of base mismatching, the combination of parameters is more important than the absolute measure of any one factor. Examples of relevant hybridization conditions include (in order of increasing stringency): incubation temperatures of 25° C., 30° C., 35° C., and 37° C.; buffer concentrations of 10× SSC, 6× SSC, 1× SSC, 0.1× SSC (where SSC is 0.15 M NaCl and 15 mM citrate buffer) and their equivalents using other buffer systems; formamide concentrations of 0%, 25%, 50%, and 75%; incubation times from 5 minutes to 24 hours; 1, 2, or more washing steps; wash incubation times of 1, 2, or 15 minutes; and wash solutions of 6× SSC, 1× SSC, 0.1× SSC, or deionized water.

“Opposing ends of probes,” and variations thereof, as used herein, refer to ends of a donor-acceptor probe pair that would be closest to each other when the donor probe and acceptor probe are hybridized to a target polynucleotide (e.g., 5′ end of one probe and 3′ end of the other probe).

A “mismatch site,” as used herein, refers to a site in a probe or target polynucleotide that is non-complementary with the corresponding site in a target polynucleotide or probe, respectively. A mismatch site can comprise one or more nucleotides.

A “portion” or “region,” used interchangeably herein, of a probe or polynucleotide is a sequence of at least one base. In some embodiments, a region or portion is at least about 1, 3, 5, 10, 15, 20, 25 contiguous nucleotides.

“Fluorescence resonance energy transfer” (“FRET”) is a physical phenomenon that occurs between two fluorophores when they are in physical proximity to one another and the emission spectrum of one fluorophore overlaps the excitation spectrum of the other, and wherein the emission of one fluorophore provides excitation energy for the other fluorophore.

“A”, “an” and “the”, and the like, unless otherwise indicated include plural forms.

“Comprising” means including.

Conditions that “allow” an event to occur or conditions that are “suitable” for an event to occur, such as hybridization and the like, or “suitable” conditions are conditions that do not prevent such events from occurring. Thus, these conditions permit, enhance, facilitate, and/or are conducive to the event. Such conditions, known in the art and described herein, depend upon, for example, the nature of the nucleotide sequence, temperature, and buffer conditions. These conditions also depend on what event is desired, such as hybridization, generation or detection of a signal.

Sequence “mutation,” as used herein, refers to any sequence alteration in a sequence of interest in comparison to a reference sequence. A reference sequence can be a wild type sequence or a sequence to which one wishes to compare a sequence of interest. A sequence mutation includes single nucleotide changes, or alterations of more than one nucleotide in a sequence, due to mechanisms such as substitution, deletion or insertion. Single nucleotide polymorphism (SNP) is also a sequence mutation as used herein.

“Microarray” and “array,” as used interchangeably herein, comprise a surface with an array, preferably an ordered array, of putative binding (e.g., by hybridization) sites for a biochemical sample (target) which often has undetermined characteristics. In a preferred embodiment, a microarray refers to an assembly of distinct polynucleotide or oligonucleotide probes immobilized at defined positions on a substrate. Arrays are formed on substrates fabricated with materials such as paper, glass, plastic (e.g., polypropylene, nylon), polyacrylamide, nitrocellulose, silicon, optical fiber or any other suitable solid or semi-solid support, and configured in a planar (e.g., glass plates, silicon chips) or three-dimensional (e.g., pins, fibers, beads, particles, microtiter wells, capillaries) configuration. Probes forming the arrays may be attached to the substrate by methods described in co-pending U.S. patent application Ser. Nos. 09/758,873 and 09/938,798, which are hereby incorporated in their entirety by reference. Other methods include (i) in situ synthesis (e.g., high-density oligonucleotide arrays) using photolithographic techniques (see, Fodor et al., Science (1991), 251:767-773; Pease et al., Proc. Natl. Acad. Sci. U.S.A. (1994), 91:5022-5026; Lockhart et al., Nature Biotechnology (1996), 14:1675; U.S. Pat. Nos. 5,578,832; 5,556,752; and 5,510,270); (ii) spotting/printing at medium to low-density (e.g., CDNA probes) on glass, nylon or nitrocellulose (Schena et al, Science (1995), 270:467-470, DeRisi et al, Nature Genetics (1996), 14:457-460; Shalon et al., Genome Res. (1996), 6:639-645; and Schena et al., Proc. Natl. Acad. Sci. U.S.A. (1995), 93:10539-11286); (iii) masking (Maskos and Southern, Nuc. Acids. Res. (1992), 20:1679-1684) and (iv) dot-blotting on a nylon or nitrocellulose hybridization membrane (see, e.g., Sambrook et al., Eds., 1989, Molecular Cloning: A Laboratory Manual, 2nd ed., Vol. 1-3, Cold Spring Harbor Laboratory (Cold Spring Harbor, N.Y.)). Probes may also be noncovalently immobilized on the substrate by hybridization to anchors, by means of magnetic beads, or in a fluid phase such as in microtiter wells or capillaries.

The term “probe portion” (or “probe component”) as used herein, refers to a nucleic acid molecule or a set of copies of a nucleic acid molecule which is capable of specific binding to a nucleic acid molecule(s) in a particular sample or portion of a sample. The set may contain any number of copies of the nucleic acid molecule. A “probe portion” (or “probe component”) may be a protein that is either formed in situ on a substrate using known techniques or a protein that is attached to a substrate using known techniques. A “probe portion” (or “probe component”) may also be a cell derived from or found in a living entity such as a plant or animal. “Probes,” as used herein, refers to more than one probe. Probes may be immobilized on a substrate by either covalent or noncovalent attachment. Probes may be immobilized on the substrate indirectly, for example through being linked to a spacer element that is in turn linked to the substrate.

A “solid substrate,” as used herein, includes materials such as paper, glass, plastic (e.g., polypropylene, nylon), polyacrylamide, nitrocellulose, silicon, optical fiber, or any other suitable solid or semi-solid support. The solid substrate can be configured in a planar (e.g., glass plates, silicon chips) or three-dimensional (e.g., pins, fibers, beads, particles, microtiter wells, capillaries) configuration.

A “spacer,” as used herein, refers to a component of a probe of the invention that links the probe component to a solid substrate. The spacer is included in the probes of the invention to, for example, extend the distance between the nucleic acid backbone and the solid substrate on which the probe is immobilized. A spacer preferably comprises a substance that is not hybridizable by a target polynucleotide. Suitable such substances include polyethylene glycol and deoxyribothymidine (dT).

A “minor groove binder,” or “MGB,” as used herein, refers to a molecule that is able to fit into the minor groove of the helix formed by double-stranded nucleic acids, generally and preferably double-stranded DNA, thereby stabilizing nucleic acid duplexes. A MGB may be a naturally-occurring molecule, such as an antibiotic, or a synthetic molecule.

The terms “linked,” and “attached,” and variations thereof, as used herein, refer to a direct or indirect connection between two elements (for example, a spacer and a nucleic acid, or a spacer and a solid substrate), wherein the connection can be formed by covalent or noncovalent means, such as via biotin and avidin or streptavidin.

A component is linked to a nucleic acid “at one end” or “at the end” when it is linked to the last nucleotide at one end of the nucleic acid. A component is linked to another component “near one end” or “near the end” of that component. A nucleotide that is near the last nucleotide is preferably fewer than about 9 nucleotides, more preferably fewer than about 6 nucleotides, even more preferably fewer than about 3 nucleotides, and most preferably about 1 nucleotide. A spacer is linked to e.g. a protein or cell “at one end” or “at the end” when the spacer is linked to a site on the protein or cell that allows the protein or cell or portion thereof to be free of the surface of an immobilizing substrate to which the cell or protein is attached when in use. A spacer is “near one end” or “near the end” when it is linked sufficiently closely to “the end” as described above that the cell or protein has a particular portion of interest spaced a sufficient distance from the surface of an immobilizing substrate during use that the portion is available for binding.

“Oligonucleotide,” as used herein, generally refers to short, generally single stranded, generally synthetic polynucleotides that are generally, but not necessarily, less than about 200 nucleotides in length. Oligonucleotides in the present invention include the nucleic acid backbone of probes. The terms “oligonucleotide” and “polynucleotide” are not mutually exclusive. The description above for polynucleotides is equally and fully applicable to oligonucleotides.

A “synthetic molecule,” as used herein, refers to a molecule that can be synthesized, usually chemically, which generally does not exist in nature. Examples of a synthetic molecule include, but are not limited to, peptides and polynucleotides.

Methods for Large Scale Detection and Quantification of Nucleic Acid Sequences Using Pairs of Probes with Interactive Signaling Moieties

Donor and Acceptor Probes

Probes of the invention can be prepared using any of a variety of methods known in the art. For example, a probe can be generated by chemical synthesis common the in the art of oligonucleotide synthesis. A donor or acceptor moiety can be incorporated into a probe by methods known in the art. For example, a nucleotide to which an acceptor or donor moiety is attached can be provided in the synthesis reaction, such that a probe is synthesized to contain a nucleotide containing the acceptor or donor moiety. Synthesis reactions can be modified as necessary according to methods known in the art to incorporate these moieties at desired locations in the probe. In another example, a probe can be treated post-synthesis with acceptor or donor moieties under conditions known in the art to effect attachment of said moieties to nucleotides of the probe. Methods for synthesizing labeled probes are described in, for example, Agrawal and Zamecnik, Nucl. Acids. Res. (1990), 18(18):5419-5423; MacMillan and Vetdine, J. Org. Chem. (1990), 55:5931-5933; Pieles et al., Nucl. Acids. Res. (1989), 17(22):8967-8978; Roger et al., Nucl. Acids. Res. (1989), 17(19):7643-7651; Fisher and Watson, U.S. Pat. No. 5,491,063; and Tesler et al., J. Am. Chem. Soc. (1989), 111 :6966-6976. A review of synthesis methods is provided in, for example, Goodchild, Bioconjugate Chemistry (1990), 1(3):165-187.

In embodiments of the invention, acceptor and donor moieties can be attached to separate polynucleotides or to the same polynucleotide. In one embodiment, an acceptor or donor moiety is located at the 5′ end of a probe. In another embodiment, an acceptor or donor moiety is located at the 3′ end of a probe. In yet another embodiment, an acceptor or donor moiety is located in an internal position in the probe (i.e., attached to an internal nucleotide). In a probe containing a mismatch site (or suspected of containing the mismatch site), the distance between the mismatch site and the moiety of the probe is preferably about 15 nucleotides or less, more preferably less than about 12 nucleotides, even more preferably less than about 9 nucleotides, and most preferably less than about 6 nucleotides. In another embodiment, the donor and acceptor moieties are attached to a single polynucleotide of at least about 25-200, 50-150, or 60-100 nucleotides in length.

The probe (either donor or acceptor) that spans the sequence of interest is preferably designed such that any mismatch site is located sufficiently towards the probe end that has an opposing end in the co-hybridized probe. The portion of the probe between the mismatch site and said probe end should be of a sufficiently short length that its hybridization to the target polynucleotide is substantially inhibited. There is substantial inhibition when there is a reduction of preferably at least about 50%, more preferably at least about 70%, even more preferably at least about 90%, still more preferably at least about 95%, and most preferably at least about 100% (i.e. no hybridization) reduction in hybridization of the portion of the probe to its complementary sequence in the target polynucleotide. The length of the portion of the probe is preferably less than about 12 nucleotides, more preferably less than about 9 nucleotides, even more preferably less than about 6 nucleotides, and most preferably less than about 3 nucleotides. The length of the remaining portion of the probe is generally selected to ensure that this portion of the probe is hybridizable to a target polynucleotide even under stringent hybridization conditions. Examples of suitable lengths of the remaining portion of the probe include, but are not limited to, those that are preferably more than about 10, 15, 20, 25 or 30 nucleotides. The length of a co-hybridized probe is generally selected to ensure its hybridization with a target polynucleotide even under stringent hybridization conditions. Said length is preferably at least about 15 nucleotides, more preferably at least about 20 nucleotides, even more preferably at least about 25 nucleotides, and most preferably at least about 30 nucleotides.

Various combinations of donor-acceptor moieties, such as those capable of energy transfer when in close spatial proximity, can be used. In a preferred embodiment, the donor-acceptor moieties are capable of fluorescence resonance energy transfer (FRET). FRET is a dipole-dipole coupling process by which the excited-state energy of a fluorescent donor molecule is non-radiatively transferred to an unexcited acceptor molecule over distances of, e.g., 10-80 Å. The occurrence of FRET results in a quenching of donor fluorescence and an enhancement of acceptor fluorescence intensity. The rate constant for FRET by a resonance mechanism is dependent, among other factors, on the separation distance between the donor-acceptor pair; the constant is inversely proportional to the sixth power of the distance. Consequently, FRET can also be used as a way for determining the distance between a donor and an acceptor molecule. FRET is further discussed in U.S. Pat. No. 6,140,054. Suitable fluorophores include, but are not limited to, fluorescein/rhodamine, phycoerythrin/Cy7, fluorescein/Cy5 or fluorescein/Cy5.5. Various other suitable donor-acceptor moieties capable of FRET are described in, for example, U.S. Pat. No. 4,996,143 (e.g., fluorescein and Texas Red donor acceptor dye pair), and U.S. Pat. No. 5,688,648.

Various other donor-acceptor moieties are known in the art, and can be used in the present invention. For example, the moieties may be ligands for reporter molecules which can interact with each other when brought in close spatial proximity, the interaction of which prevents or enables activity of one of the reporter molecules. Examples for suitable combinations of reporter groups useful for the methods of the invention are enzyme-inhibitor combination, reporter molecules which when reacting with one another form an active enzyme molecule, and the like. The dissociation of the two interacting reporter groups is detectable and indicative of the presence of one or more nucleic acid sequence(s) of interest in a sample, the quantity of nucleic acid sequence(s) of interest in a sample or the identity of a nucleic acid sequence by comparison to that of a reference nucleic acid sequence. Another example of a donor-acceptor pair is an enzyme and its corresponding substrate or two different portions or subunits of an enzyme that catalyze a detectable reaction, such that when both moieties are in close enough proximity, the enzyme is capable of catalysis of a substrate to form a detectable product and when the moieties are not in close enough proximity for catalysis, a diminished amount of detectable reaction product is produced.

Detecting and/or Quantifying a Sequence of Interest

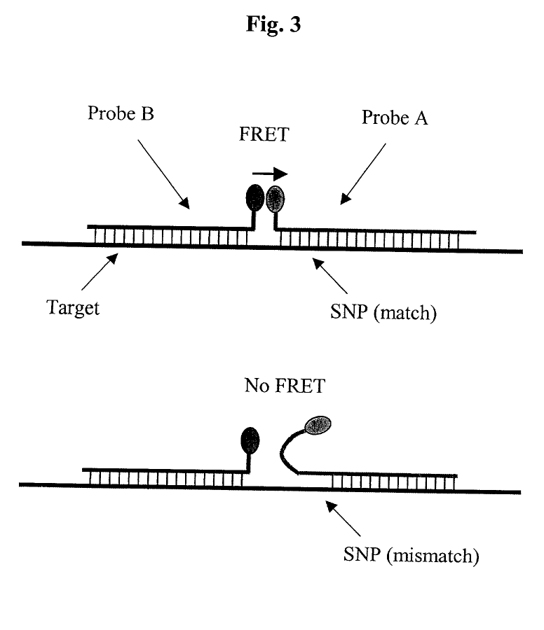

In the methods of the invention, two oligonucleotides are used as a pair of probes to detect a nucleic acid sequence of interest. One illustrative embodiment of the methods of the invention is illustrated in FIG. 3. As shown in FIG. 3, one probe is labeled with an acceptor fluorescence molecule (probe A) and has a sequence hybridizing to a specific site of a target polynucleotide. The other is labeled with a donor fluorescence molecule (probe B) and has a sequence hybridizing to an adjacent site of the target polynucleotide where the first probe hybridizes. When the two probes are hybridized to a target polynucleotide adjacent to each other, the distance between the two fluorescence molecules (acceptor and donor moieties) become spatially close on the formed hybrid, allowing FRET to occur, resulting in changes in the fluorescence spectrum leading to a detectable signal.

Methods of the invention can be used for genotyping. In one illustrative embodiment of a genotyping scheme using methods of the invention, an allele-specific oligonucleotide probe (such as probe A in FIG. 3) serves as the acceptor probe whereas an co-hybridized, preferably adjacent, oligonucleotide probe (such as probe B in FIG. 3) serves as the donor. When both probes are hybridized to a target polynucleotide, the donor and the acceptor moieties (such as fluorescence molecules) are located such that the distance between them allows efficient energy transfer. The distance can be a number of nucleotides apart, as determined by the spectral properties of the two fluorescence molecules and their Forster radius (R0). In the embodiment illustrated in FIG. 3, the donor and acceptor moieties are depicted as attached to the 5′ and 3′ end of oligonucleotide probes, respectively. As described above, both moieties could also be attached to internal nucleotides of the oligonucleotide probes. Under stringent hybridization conditions, both the allele-specific oligonucleotide probe and the co-hybridized corresponding probe of the donor-acceptor probe pair would be expected to be capable of hybridizing to the target polynucleotide. If the allele-specific oligonucleotide probe contains a polymorphic nucleotide that matches with the target sequence (i.e., no mismatch), the sequence in probe A that is hybridizable to the sequence of interest in the target polynucleotide will hybridize to said sequence and the acceptor moiety will be in close proximity with the donor moiety, resulting in a significant interaction between the donor and acceptor moieties, and thus a significant FRET response (if fluorescence molecules are used). If, however, the allele-specific probe A contains a polymorphic nucleotide that mismatches with the sequence of interest, under the same stringent hybridization condition, the portion of probe A between the mismatch nucleotide and the acceptor moiety at the 3′ end will not hybridize with the target polynucleotide, resulting in increased distance between the donor and acceptor moieties and diminished interaction, and thus for example diminished FRET response (if fluorescence molecules are used). Diminished interaction of the moieties, resulting for example in a difference in FRET response, serves as the basis for genotyping using methods of the invention. In this illustrative embodiment, the distance between the polymorphic nucleotide and the acceptor moiety at the 3′ end is preferably about 15 nucleotides or less, more preferably less than about 12 nucleotides, more preferably less than about 9 nucleotides, more preferably less than about 6 nucleotides, so as to ensure that the polymorphic mismatch inhibits hybridization of the portion of the probe between the mismatch site and the 3′ end and thus ensuring that the difference in interaction mode of the moieties (such as FRET response) between match and mismatch remains distinguishable. In some embodiments, the distance between the polymorphic nucleotide and the acceptor moiety at the 3′ end is 1, 2, 3, 4, 5, 6, 7, 8, 9, 10, 11, 12, 13, 14, or 15 nucleotides. In this illustrative embodiment, the distance between the polymorphic nucleotide and the 5′ end of the probe is preferably more than about 15 nucleotides, more preferably more than about 20 nucleotides, more preferably more than about 25 nucleotides, more preferably more than about 30 nucleotides, so as to ensure that this part of the probe is hybridized with the target polynucleotide even under stringent hybridization conditions.

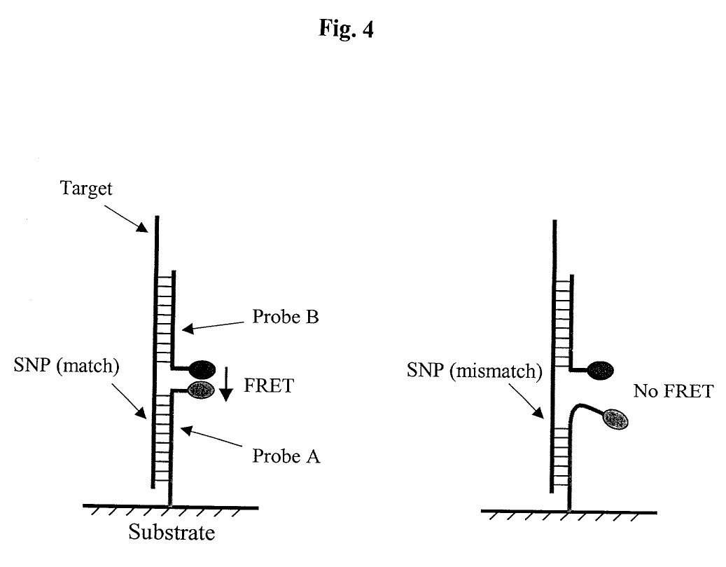

Methods of the invention can be adapted to an array format, where, for example, allele-specific oligonucleotide probes with acceptor fluorescence molecules are attached to a solid surface and the adjacent co-hybridized probes with donor fluorescence molecules remain in solution phase, thus allowing potentially thousands of sequence analyses in parallel (as illustrated in FIG. 4). Suitable apparatuses for forming arrays include those described in co-pending patent applications entitled “Linear Probe Carrier”, U.S. Ser. No. 09/758,873, filed Jan. 10, 2001, “Three-Dimensional Probe Carriers”, U.S. Ser. No. 09/938,798, “Microarray Fabrication Technologies,” U.S. Ser. No. 09/791,994, filed Feb. 22, 2001, and “Microarray Fabrication Techniques and Apparatus”, U.S. Ser. No. 09/791,998, also filed Feb. 22, 2001.

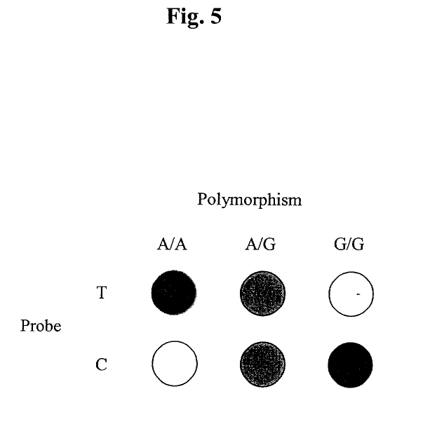

In one embodiment, allele-specific oligonucleotide probes with acceptor fluorescence molecules are located in different spots on an array substrate. When they are contacted with samples containing homozygous or heterozygous genotypes, the probes would show a distinct pattern of FRET response. FIG. 5 depicts a schematic diagram of genotype array for detection of a single nucleotide polymorphism. The top and bottom spots are designed to contain probes complementary to the allelic sequences containing A and G, respectively. As illustrated in FIG. 5, samples containing homozygous A and G alleles result in the top and bottom spots turning black, respectively; samples containing alleles that do not match a probe result in white spots; and samples containing heterozygous A/G result in gray spots.

Methods of the invention can also be used for gene expression profiling. In this aspect, methods of the invention are conducted using samples containing 2 or more RNA sequences of interest, or derivatives thereof (for example, CDNA generated from RNA). The methods can be performed to determine the presence of and/or to quantify multiple species of RNA (or derivatives thereof) that represent the gene expression profile in a sample. Generally, such a sample would be obtained from a single source, which would be the source for which gene expression profiling is desired.

Methods of the invention can be performed in liquid format where the hybridization kinetics can be monitored in real time. A vessel that will allow highly parallel sample handling in liquid phase while also facilitating signal detection is the GeneHive microwell design (described in co-pending U.S. application Ser. No. 09/938,798). The signal can be detected with either a scanner equipped with temperature controlled staged or a CCD camera. In yet another process, fluorescently labeled probes can be attached to flat solid surfaces as in traditional DNA microarrays, and hybridization reactions are permitted to occur under a coverslip while being monitored with a scanner or CCD camera. Such real time monitoring is only possible with a system such as the present invention, which does not require washing prior to signal detection. Since the temperature change permits the real time monitoring of energy transfer under a continuum of hybridization stringencies, no optimization of the probes is required for each gene to match the Tm.

Nucleic Acid Probes with Enhanced Hybridization Efficiency and Specificity

Probes of the Invention

The probes discussed below are provided by way of example. The nucleic acid probes discussed below may alternatively be protein probes or cellular probes as described herein.

The present invention provides nucleic acid probes with enhanced hybridization efficiency and specificity. This is achieved by including a spacer element between the nucleic acid backbone of a probe and the solid substrate to which the probe is to be immobilized. We have found that increasing the distance of the nucleic acid backbone of probes from the surface of a solid support through the use of a spacer element (such as a spacer comprising either deoxyribothymidine or polyethylene glycol) greatly increases hybridization signals on DNA microarrays. (See FIGS. 2A & B.) Longer spacers are generally more efficient in signal enhancement. The inclusion of a minor groove binder (MGB) molecule into the nucleic acid backbone of probes also contributes to enhanced hybridization efficiency and specificity of the probes of the invention. In a preferred embodiment, probes of the invention comprise a spacer element between the nucleic acid backbone and a solid support, and a minor groove binder molecule. By combining the use of spacers and MGBs, one not only increases the hybridization efficiency, and therefore the sensitivity of microarray-based analysis, but also the discriminating power between perfect sequence matches and mismatches, and therefore the specificity of gene expression or genotyping systems.

Accordingly, probes of the invention comprise a nucleic acid backbone separated from the surface of a solid support by a spacer. In addition to providing a linker function between the nucleic acid backbone and a solid support, the spacer element in these probes also functions as a size exclusion molecule to reduce background binding and increase hybridization efficiency. In one embodiment, the spacer comprises varying numbers of deoxyribothymidine residues (dT). Deoxyribothymidine, and similar compounds, are particularly suitable for use as a spacer because many target polynucleotides (such as a sequence of interest) are generated using oligo-dT as a primer, for example in the generation of first strand cDNA. These target polynucleotides would not hybridize efficiently with a spacer comprising dT residues. In addition, T:A base paring is less stable than G:C base paring, thereby reducing the possibility of non-specific binding of target molecules to deoxyribothymidine-containing spacer sequences. The length of a dT spacer can be any length that permits linking between a solid substrate and the nucleic acid backbone while increasing the liquid hybridization kinetics of the probe above the kinetics observed when the nucleic acid backbone of a probe is attached to a solid substrate without a dT spacer. The length of a dT spacer is preferably from about 1 to about 20 dT residues. It is preferably at least about 1, more preferably at least about 3, even more preferably at least about 9, still more preferably at least about 12, more preferably at least about 15, and most preferably at least about 20 dT residues.

In another embodiment, the spacer comprises inert compounds such as polyethylene glycol (PEG). PEG molecules, either monomers or oligomers (e.g., 18 monomer units) can be attached to the nucleic acid backbone of probes during synthesis of the nucleic acid backbone. The length of a PEG spacer can be any length that permits linking between a solid substrate and the nucleic acid backbone while increasing the liquid hybridization kinetics of the probe above the kinetics observed when the nucleic acid backbone of a probe is attached to a solid substrate without a PEG spacer. The length of a PEG spacer is preferably at least about 3, more preferably at least about 6, even more preferably at least about 9, still more preferably at least about 12, more preferably at least about 18, and most preferably at least about 25 polyethylene glycol monomers.

In a preferred embodiment, probes of the invention also include a MGB molecule. MGBs can be attached to the 3′-end, 5′-end or to an internal nucleotide of the nucleic acid backbone of the probes of the invention. A probe can have one or more MGB molecules attached to it. The number of MGB molecules per probe depends in part on the length of the nucleic acid backbone of the probe. The number of MGB molecules (as a percentage based on number of MGB molecules versus number of nucleotides in the nucleic acid backbone) in each probe is preferably at least about 0.5%, more preferably at least about 2%, even more preferably at least about 5%, still more preferably at least about 10%, and even more preferably at least about 20%. Various MGBs are known in the art, including, for example, naturally occurring antibodies and synthetic molecules, such as PNU166196 (Geroni et al., Clinical Cancer Research (suppl.), Volume 6, November 2000) and those described in U.S. Pat. No. 6,084,102. Use of MGBs for enabling DNA analysis by stabilizing DNA duplexes and increasing mismatch discrimination characteristics when conjugated to an oligonucleotide probe has been described, for example by Epoch Biosciences (see www.epochbio.com). MGB-conjugated oligonucleotides are reported to increase melting temperatures (Tm) of probe-DNA duplexes, allow shorter probes to be used, allow better mismatch discrimination, allow mismatch discrimination over a wide range of temperatures, have low background fluorescence, produce Tm leveling, provide improved mismatch discrimination when the mismatch is at the same position as the MGB, and be useful in sequence-specific clamping. Epoch's MGB technology is reported to improve existing hybridization-based detection methods, for use in applications such as genetic analysis, gene expression and identification of infectious organisms. Epoch's MGB technology can also be used in oligonucleotides immobilized on solid supports. Such immobilized oligonucleotides have been used in detection of single nucleotide polymorphisms for two BCL2 alleles. (See www.epochbio.com.)

The length of the nucleic acid backbone of probes of the invention can be any length that is suitable for the hybridization reactions desired. The length is preferably from about 10 to about 80 nucleotides, more preferably from 20 to 60 nucleotides, even more preferably from about 30 to about 50 nucleotides, and most preferably from about 40 to about 45 nucleotides.

Probes of the invention can be prepared using any of a variety of methods known in the art. For example, the nucleic acid backbone of the probes in which an MGB is incorporated can be generated by synthesis methods common in the art of oligonucleotide synthesis, and as described in the art, such as for example at www.epochbio.com. Minor groove binders can be attached to the nucleic acid during synthesis of the nucleic acid on a commercial synthesizer or post-synthetically using reagents and methods known in the art. (See www.epochbio.com.) For example, a nucleotide to which an MGB molecule is attached can be provided in the synthesis reaction, such that a nucleic acid molecule (which serves as the nucleic acid backbone of the probe) is synthesized to contain a nucleotide containing the MGB. Synthesis reactions can be modified as necessary, according to methods known in the art, to incorporate MGBs at desired locations in the probe. In another example, a nucleic acid molecule can be treated post-synthetically with MGB molecules under conditions known in the art to effect attachment of molecules to nucleotides of the nucleic acid molecule. A review of synthesis methods is provided in, for example, Goodchild, Bioconjugate Chemistry (1990), 1(3):165-187.

Methods of synthesis of probes with dT spacers are known in the art. For example, synthesis of nucleic acid backbone molecules with oligo-dT molecules attached to one end can be achieved using routine methods known in the art of oligonucleotide synthesis. Attachment of the oligo-dT linker (spacer) to a solid substrate can be achieved using various methods known in the art, for example those used in attachment of oligonucleotide probes to solid substrates, for example as described in Southern et al., Nat. Gen. Supp. (1999), 21:5-9 and co-pending applications entitled “Linear Probe Carrier” and “Three-Dimensional Probe Carriers” which are hereby incorporated in their entirety by reference.

Methods of synthesis of probes with PEG spacers include those described above for attaching MGB molecules to the nucleic acid backbone. For example, nucleotides to which a PEG molecule is attached can be included in the synthesis of a nucleic acid backbone of a probe. Alternatively, PEG can be used to functionalize glass surfaces. (Southern et al., supra.) For example, one end of a PEG oligomer can be attached to the surface of a solid substrate. The other end (the solution end) of the oligomer can have a chemically active functional group, such as an acetyl, amino, or thiol group, which will allow the nucleic acid backbone of the probes of the invention to be attached to the PEG oligomer through a corresponding chemical group on a nucleotide in the nucleic acid backbone.

Probes can be attached to a solid support through any of a number of methods and/or mechanisms known in the art. For example, amino-modified nucleic acids (such as DNA, for example oligo-dT) can be attached to an aldehyde-functionalized surface via reaction with free aldehyde groups using Schiff's base chemistry. In another example, amino-terminal nucleic acids can be coupled to isothiocyanate-activated glass, to aldehyde-activated glass, or to a glass surface modified with epoxide. In yet another example, carboxylated or phosphorylated nucleic acids can be coupled on aminated supports, or aminated nucleic acids can be coupled on carboxylated or phosphorylated supports.

In some embodiments, probes of the invention comprise labels. Methods for generating probes with labels are known in the art. For example, nucleotides (such as deoxyribonucleoside triphosphates) to which a desired label is attached can be used in the synthesis of the nucleic acid backbone to generate a labeled nucleic acid product. Labels suitable for use in the probes of this invention are known in the art, and include, for example, fluorescent dye labels. Generally, suitable labels for probes of the invention are capable of generating a detectable signal upon interaction with a counterpart label provided by another sequence, for example a sequence of interest to which a probe is hybridized (e.g., a donor-acceptor pair, as described above). Thus, generally, if probes of the invention are labeled, homogeneous detection of hybridization to a nucleic acid of interest can be used. For example, the optical properties of a label associated with a probe of the invention can be altered subsequent to hybridization of the probe to a sequence of interest (e.g., such a label includes fluorescent dyes that undergo fluorescence resonance energy transfer (FRET) when donor and acceptor fluorescent moieties are placed in sufficient proximity to each other, for example as described above). Other label combinations are also possible. For example, two ligands (such as digoxigenin and biotin) each attached to a probe of the invention and a sequence of interest, respectively, can be brought into close proximity in the context of hybridization of the probe to the sequence of interest. Binding of the two ligands with their corresponding antibodies which are differentially labeled can be detected due to the interaction of the labels on the antibodies. For instance, if the two different labels are a photosensitizer and a chemiluminescent acceptor dye, the interaction of the labels can be detected by the luminescent oxygen channeling assay as described in U.S. Pat. No. 5,340,716.

Probes of the Invention in Array Format

Probes of the invention are particularly useful for making arrays, as they address some of the most serious problems in the use of array technology. The enhanced hybridization features of the probes, resulting in part from the enhancing liquid hybridization kinetics characteristics of probes attached to a solid substrate, provides a significant advantage in making improved microarrays. Suitable apparatuses for forming arrays of probes of the invention include those described in co-pending U.S. patent application Ser. Nos. 09/758,873 and 09/938,798.

Probes of the invention, particularly when provided as microarrays, are useful in a number of nucleic acid analysis applications. For example, they can be used in genotyping and gene expression profile analysis, using detection and quantification methods known in the art. The probes can be used in the methods described above for detection and quantification of nucleic acid sequences using pairs of probes with interactive signaling moieties.

### EXAMPLES

The following example is provided to illustrate but not limit the present invention.

### Example 1

### Liquid Phase Hybridization of a FRET Probe Pair to a Target and Detection of Interaction Between the Probes

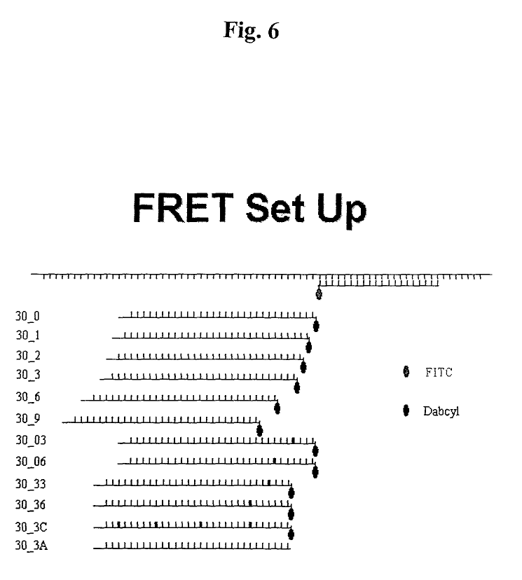

A real-time PCR system from ABI (ABI 7700) was used to assess the interaction between a FRET probe pair. One probe in the probe pair (contained a FITC label at the 5′ end. A set of probes containing a Dabcyl quencher molecule at the 3′ end (FIG. 6) constituted the other probe of the probe pair. Probes in this set varied in distance from the FITC label when the probe pairs were hybridized to a target, thereby placing the Dabcyl quencher 0, 1, 2, 3, 6, or 9 bases away from the FITC molecule, or by placement of a mismatch at varying distances from the Dabcyl molecule (FIG. 6).

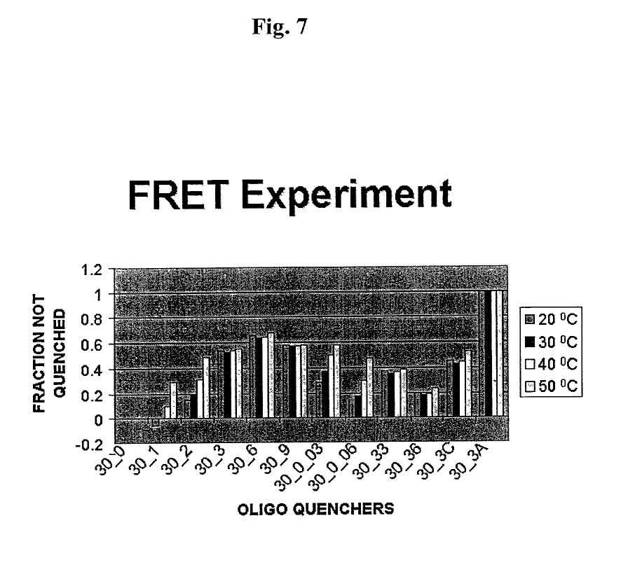

When equimolar amounts of the FITC probe and a 70-mer target were mixed with an excess amount of one of the quencher probes from the above-mentioned set, and the mixture subjected to heat-denaturing and cooling cycles, the quenching of the FITC signal was affected by the distance between the FITC and Dabcyl molecules. Under all temperatures tested, the strongest quenching effect was observed when the quencher and the dye molecules were 0 bases apart (FIG. 7). Significantly in the present of a mismatch 3 or 6 bases away from the quencher molecule, the quenching effect was reduced by as much at 60% (columns labeled “30 0 03” and “30 0 06” in FIG. 7).

The results indicate that the dye and quenching molecules must be in close proximity for the quenching effect to occur. The reduced quenching in the presence of mismatches suggests that this method can be used for genotyping of single nucleotide polymorphisms.

Although the foregoing invention has been described in some detail by way of illustration and examples for purposes of clarity of understanding, it will be apparent to those skilled in the art that certain changes and modifications may be practiced without departing from the spirit and scope of the invention. Therefore, the description should not be construed as limiting the scope of the invention, which is delineated by the appended claims.

All publications, patents, and patent applications cited herein are hereby incorporated by reference in their entirety for all purposes to the same extent as if each individual publication, patent, or patent application were specifically and individually indicated to be so incorporated by reference.

We claim:

1. A method of detecting a sequence of interest in a sample, said method comprising contacting the sample with an acceptor probe and a donor probe,
wherein the acceptor and donor probes comprise polynucleotides that are hybridizable to non-overlapping portions of a target polynucleotide, wherein the acceptor probe comprises a sequence hybridizable to the sequence of interest,
wherein said acceptor probe comprises an acceptor moiety and said donor probe comprises a donor moiety, and wherein hybridization of both the donor and acceptor probes to a target polynucleotide comprising the sequence of interest allows the donor moiety and the acceptor moiety to interact, wherein the interaction is detectable, and
wherein a mismatch between the sequence of interest and the acceptor probe substantially prevents a portion of the acceptor probe from hybridizing to the sequence of interest such that the interaction between the donor and acceptor probe is diminished, and wherein said portion of the acceptor probe is the portion that spans the sequence of the target polynucleotide between the mismatch site and hybridization site of the opposing end of the donor probe.

2. A method of detecting a sequence of interest in a sample, said method comprising contacting the sample with an acceptor probe and a donor probe,
wherein the acceptor and donor probes comprise polynucleotides that are hybridizable to non-overlapping portions of a target polynucleotide, wherein the donor probe comprises a sequence hybridizable to the sequence of interest,
wherein said acceptor probe comprises an acceptor moiety and said donor probe comprises a donor moiety, and wherein hybridization of both the donor and acceptor probes to a target polynucleotide comprising the sequence of interest allows the donor moiety and the acceptor moiety to interact mode, wherein the interaction is detectable, and
wherein a mismatch between the sequence of interest and the donor probe substantially prevents a portion of the donor probe from hybridizing to the sequence of interest such that the interaction of the donor moiety and the acceptor moiety is diminished, and wherein said portion of the donor probe is the portion that spans the sequence of the target polynucleotide between the mismatch site and hybridization site of the opposing end of the acceptor probe.

3. The method of claim 1, wherein said sequence of interest comprises a mutation, wherein said mutation is selected from the group consisting of a point mutation, a deletion, and an insertion.

4. The method of claim 2, wherein said sequence of interest comprises a mutation, wherein said mutation is selected from the group consisting of a point mutation, a deletion, and an insertion.

5. The method of claim 1, wherein said sequence of interest comprises a single nucleotide polymorphism.

6. The method of claim 2, wherein said sequence of interest comprises a single nucleotide polymorphism.

7. A method of quantifying a sequence of interest in a sample, comprising quantifying the detectable interaction between the donor and the acceptor moieties in the method of claim 1.

8. A method of quantifying a sequence of interest in a sample, comprising quantifying the detectable interaction between the donor and the acceptor moieties in the method of claim 2.

9. A method of determining a gene expression profile in a sample, comprising (i) detecting the presence of two or more sequences of interest in said sample using the method of claim 1; and (ii) quantifying the detectable interaction between donor and acceptor moieties for each of said sequences of interest to determine the amount of each sequence of interest present in said sample.

10. A method of determining a gene expression profile in a sample, comprising (i) detecting the presence of two or more sequences of interest in said sample using the method of claim 2; and (ii) quantifying the detectable interaction between donor and acceptor moieties for each of said sequences of interest to determine the amount of each sequence of interest present in said sample.

11. The method of claim 1, wherein the detectable interaction between acceptor and donor moieties is fluorescence resonance energy transfer.

12. The method of claim 2, wherein the detectable interaction between acceptor and donor moieties is fluorescence resonance energy transfer.

13. The method of claim 1, wherein the acceptor and donor moieties are located at opposing ends of the probes when hybridized to the target polynucleotide, wherein said opposing ends are the 5′ end of one probe and the 3′ end of the other probe.

14. The method of claim 2, wherein the acceptor and donor moieties are located at opposing ends of the probes when hybridized to the target polynucleotide, wherein said opposing ends are the 5′ end of one probe and the 3′ end of the other probe.

15. The method of claim 1, wherein the distance between the mismatch site and the moiety at the end of the probe hybridized to the sequence of interest is 15 nucleotides or fewer.

16. The method of claim 2, wherein the distance between the mismatch site and the moiety at the end of the probe hybridized to the sequence of interest is 15 nucleotides or fewer.

17. The method of claim 1, wherein the length of said portion of the probe that is substantially prevented from hybridizing to the sequence of interest is from about 3 to about 15 nucleotides.

18. The method of claim 2, wherein the length of said portion of the probe that is substantially prevented from hybridizing to the sequence of interest is from about 3 to about 15 nucleotides.

19. The method of claim 1, wherein the probes are provided as an array.

20. The method of claim 2, wherein the probes are provided as an array.

21. The method of claim 20, wherein the acceptor probe is attached to a solid support and the donor probe is provided in a solution phase.

22. The method of claim 20, wherein the donor probe is attached to a solid support and the acceptor probe is provided in a solution phase.

23. A nucleic acid probe comprising a nucleic acid with first and second ends, a spacer with first and second ends, and a minor groove binder (MGB), wherein the first end of the spacer is linked to the nucleic acid at or near the first end of the nucleic acid, the second end of the spacer is linked to a solid substrate, and the MGB is linked to the nucleic acid at or near the second end of the nucleic acid.
